(19) United States
(12) Patent Application Publication
(10) Pub. No.: US 2017/0354784 A1
(43) Pub. Date: Dec. 14, 2017

(54) PACKAGING FOR PRE-FILLED DRUG CARTRIDGES WITH PEG FOR PUSHING CARTRIDGE STOPPER FOR PRIMING PURPOSES

(71) Applicant: SANOFI-AVENTIS DEUTSCHLAND GMBH, Frankfurt am Main (DE)

(72) Inventors: Eva Schiendzielorz, Frankfurt am Main (DE); Katrin Nissen, Frankfurt am Main (DE)

(21) Appl. No.: 15/542,535

(22) PCT Filed: Jan. 11, 2016
(86) PCT No.: PCT/EP2016/050340

§ 371 Date: Jul. 10, 2017

(30) Foreign Application Priority Data
Jan. 12, 2015 (EP) 15150746.4

(51) Int. Cl. A61M 5/31; A61M 5/24; A61M 5/00; B65D 83/00; B65D 71/36

(52) U.S. Cl. CPC A61M 5/3146; B65D 83/0005; B65D 71/36; A61M 5/002; A61M 5/24

(57) ABSTRACT

Disclosed is a packaging for a number of pre-filled drug cartridges comprising a body to which an infusion set with tubing and needle can be fitted, the body having an open rear end, wherein a stopper is slidably disposed within the body, wherein a peg is arranged on the packaging, the peg having a height which is such that when a cartridge is being put on the peg with the rear end and pushed against it until the rear end abuts a surface of the packaging, the stopper is moved within the body by such a distance that a predetermined volume of a medicament is displaced from the cartridge for priming the cartridge and infusion set.

### CROSS-REFERENCE TO RELATED APPLICATIONS

This application is a U.S. national stage application under 35 USC §371 of International Application No. PCT/EP2016/050340, filed on Jan. 11, 2016, which claims priority to European Patent Application No. 15150746.4, filed on Jan. 12, 2015, the entire contents of which are incorporated herein by reference.

### TECHNICAL FIELD

The disclosure relates to an arrangement for priming a drug cartridge.

### BACKGROUND

When preparing an injection of a drug, e. g. by pump, a user may have to connect a tubing with a cannula to a drug cartridge. Before the tubing can be connected to an injection site such as a patient's skin, a priming shot has to be performed, i. e. a volume of the drug is displaced from the cartridge for filling the whole tubing until drug starts leaking out of the end of the tubing such that residual air is displaced from the tubing. The priming may be performed by the pump or by manually pushing a stopper within the cartridge before inserting the cartridge into the pump. As the stopper may be hard to access, the user may be tempted to use objects such as a ball pen to displace the stopper. This may result in excess displacement of drug or damage to the cartridge.

### SUMMARY

An advantage of some embodiments may be an improved arrangement for priming a drug cartridge.

An arrangement for priming a drug cartridge is provided. The drug cartridge comprises a body to which a tubing can be fitted, the body having an open rear end, wherein a stopper is slidably disposed within the body. The arrangement comprises a packaging for a number of drug cartridges, wherein a peg is arranged on the packaging, the peg having a height which is such that when a cartridge is being put on the peg with the rear end and pushed against it until the rear end abuts a surface of the packaging where the peg is located, the stopper is moved within the body by such a distance that a predetermined volume of a medicament is displaced from the cartridge. A break-loose force between the stopper and an inner wall of the body is thus overcome, i.e. the cartridge is primed, and the stopper is moved within the body. If the cartridge is then inserted into a drug delivery device, the stopper may be moved much easier as the break-loose force increases over a long storage time. This allows for specifying a pump or drive mechanism of the drug delivery device to provide a force lower than the break-loose force.

Thus, a user is enabled to prime the drug cartridge without having to resort to additional tools. Other than such tools, the peg on the packaging cannot get lost. The priming with the arrangement can be performed much faster than with a pump of a drug delivery device. Other than with additional tools, excess displacement of medicament or damage to the cartridge, e.g. a glass cartridge, is avoided.

In an exemplary embodiment, the peg has a greatest diameter at most as large as an internal diameter of the body thus avoiding friction when applying and removing the cartridge. In an exemplary embodiment, the greatest diameter of the peg is not substantially smaller than the internal diameter of the cartridge body such that the cartridge may be securely positioned on the peg and the stopper is being pushed uniformly instead of at an edge.

In an exemplary embodiment, the predetermined volume of medicament to be displaced by the priming is at least as large as an internal volume of the tubing and, if applicable, a volume of a cannula which may be fitted to the tubing. The volume is hence sufficient to fill the tubing thereby displacing air from the tubing and the cannula, if applicable.

In an exemplary embodiment, a movement D of the stopper required to displace the predetermined volume V is given by the equation

$$D = \frac{4 V}{π {B}^{2}} ,$$

wherein B is the internal diameter of the cartridge body, wherein the height of the peg is substantially equal to the movement D. This equation applies if the stopper is substantially flush with the rear end of the cartridge body in an initial state.

In an exemplary embodiment, the height of the peg can be slightly larger than the movement D in order to account for filling tolerances of the cartridges which may result in varying initial positions of the stopper in different cartridges.

If the initial position of the stopper is offset from the rear end by an offset distance, the offset distance is added to the movement D to obtain the height of the peg.

In an exemplary embodiment, the peg has a circular cylindrical shape.

In an exemplary embodiment, the diameter of the peg is smaller than the internal diameter of the cartridge body. This avoids air being trapped between the peg and the stopper when the cartridge is being put on the peg which could result in excess displacement of the stopper and consequently waste of medicament during priming, in particular if the peg and the cartridge body have the same cross section.

In an exemplary embodiment, the peg has one or more vent openings and/or notches in order to avoid the same problem. Likewise, the peg may have a non-circular cylindrical or prismatic shape, in particular a shape with a cross section differing from the cross section of the cartridge body in order to address this problem.

In an exemplary embodiment, the packaging comprises or consists of cardboard, plastics, sheet metal or wood or any other suitable material.

In an exemplary embodiment, the peg is positioned on the packaging to point upwards in a use position. This alleviates handling for the user as the cartridge may be primed with only one hand.

In an exemplary embodiment, the packaging has the shape of a box with a bottom, a cover and side walls. The peg may be arranged on the cover or on the bottom.

In an exemplary embodiment, the peg is arranged on an inner surface of the cover such that the user may open the cover, take a cartridge and prime it immediately without having to close the box first. Likewise, the peg could be arranged on an internal surface of the bottom or on an external surface of the cover.

Further scope of applicability of some embodiments will become apparent from the detailed description given hereinafter. However, it should be understood that the detailed description and specific examples, while indicating exemplary embodiments of the invention, are given by way of illustration only, since various changes and modifications within the spirit and scope of the invention will become apparent to those skilled in the art from this detailed description.

Certain aspects will become more fully understood from the detailed description given herein below and the accompanying drawings which are given by way of illustration only, and thus do not limit the present invention.

### BRIEF DESCRIPTION OF THE FIGURES

FIG. 1 is a schematic view of an exemplary embodiment of an arrangement for priming a drug cartridge comprising a packaging for drug cartridges, wherein the packaging has a peg, and

FIG. 2 is a schematic view of an exemplary embodiment of a drug cartridge and the peg.

Corresponding parts are marked with the same reference symbols in all figures.

### DETAILED DESCRIPTION

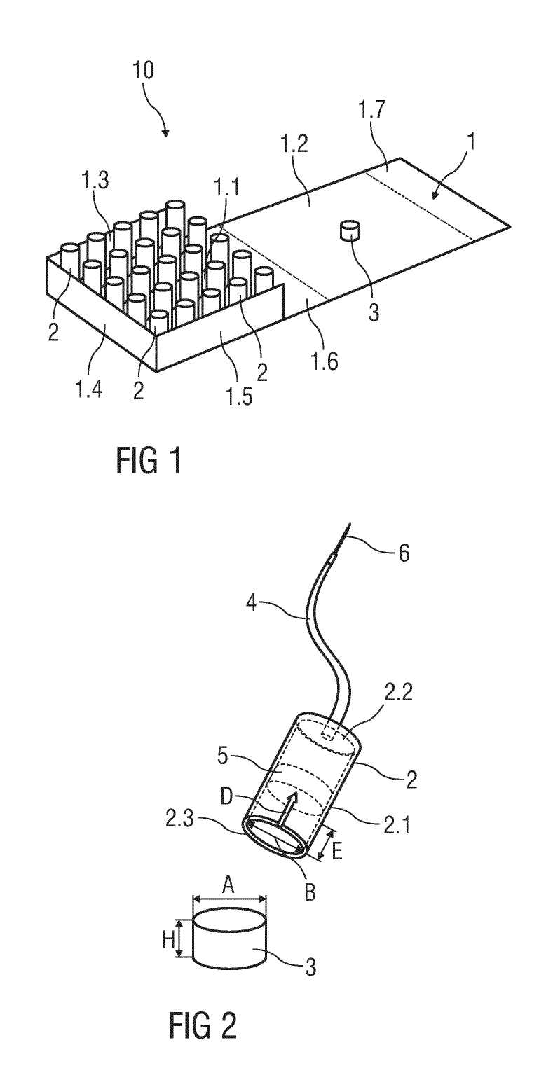

FIG. 1 is a schematic view of an exemplary embodiment of an arrangement 10 for priming a drug cartridge 2 comprising a packaging 1 for a number of drug cartridges 2.

In an exemplary embodiment, the packaging 1 has the shape of a box with a bottom 1.1, a cover 1.2 and side walls 1.3 to 1.7.

A peg 3 is arranged on an inner surface of the cover 1.2. In an alternative embodiment (not shown), the peg 3 could be provided at a different location of the package, in particular such that the peg 3 may be positioned to point upwards in a use position as shown in FIG. 1, e.g. on the cover 1.2 or on the bottom 1.1.

FIG. 2 is a schematic view of one of the drug cartridges 2 and the peg 3. The cartridge 2 comprises a cylindrical body 2.1 having a closed front wall 2.2 with an opening to which a tubing 4 can be removably fitted and an open rear end 2.3. A stopper 5 is slidably disposed within the cylindrical body 2.1. A cavity for storing a medicament, e.g. insulin, is thus defined within the cylindrical body 2.1 between the closed front wall 2.2 and the stopper 5. The stopper 5 fluid tightly seals this cavity and displaces the medicament from the cavity when being moved towards the closed front wall 2.2. In an exemplary embodiment, an end of the tubing 4 opposite the one fitted to the opening of the closed front wall 2.2 is equipped with a cannula 6 adapted to be inserted into an injection site such as a user's skin.

In an exemplary embodiment, the cylindrical body 2.1 comprises or consists of glass. In other embodiments, the cylindrical body 2.1 may comprise or consist of plastics or another suitable material.

In an exemplary embodiment, the peg 3 has the shape of a circular cylinder with a diameter A substantially corresponding to an internal diameter B of the cylindrical body 2.1. In an exemplary embodiment, the diameter A is at most as large as the internal diameter B.

The peg 3 has a height H which is such that when the cartridge 2 is being put on the peg 3 and pushed down until the rear end 2.3 abuts the surface of the cover 1.2 or any other part where the peg 3 is located, the stopper 5 is moved within the body 2.1 towards the front wall 2.2 by such a distance that a predetermined volume V of medicament is displaced from the cartridge 2 sufficient to fill the tubing 4 and the cannula 6, which a user may have attached to the cartridge 2 prior to this, thereby displacing air from the tubing 4 and the cannula 6. This action is typically referred to as priming.

The volume V of medicament to be displaced for priming depends on the internal volume of the tubing 4 and the cannula 6. The movement D of the stopper 5 required to displace this volume V is given by

$$D = \frac{4 V}{π {B}^{2}} .$$

The height H of the peg 3 may be such that a somewhat larger movement D of the stopper 5 is caused in order to account for filling tolerances of the cartridges 2 which may result in varying initial positions of the stopper 5 in different cartridges 2.

If the initial position of the stopper 5 is such that the stopper 5 is substantially flush with the rear end 2.3 of the body 2.1, the height H of the peg 3 is substantially equal to the movement D of the stopper 5. If the initial position of the stopper 5 is offset from the rear end 2.3 towards the front wall 2.2 by an offset distance E, the height H of the peg 3 is substantially equal to the sum of the movement D of the stopper 5 and the offset distance E.

In an exemplary embodiment, the amount of medicament to be displaced for priming may be 1 ml.

In an exemplary embodiment, the diameter A is smaller than the internal diameter B. This avoids air being trapped between the peg 3 and the stopper 5 when the cartridge 2 is being put on the peg 3 which could result in excess displacement of the stopper 5 and consequently waste of medicament during priming. In other exemplary embodiments, the peg 3 may have one or more vent openings and/or notches and/or the peg 3 may have a shape other than a circular cylinder, e. g. a prismatic shape with a rectangular, pentagonal, hexagonal or other polygonal cross-section to avoid air being trapped between the peg 3 and the stopper 5 when the cartridge 2 is being put on the peg 3. In any case the greatest diameter A of the peg 3 is at most as large as the internal diameter B of the body 2.1, wherein in the case of a circular cylindrical peg 3 the greatest diameter A is the only diameter A.

In an exemplary embodiment, the greatest diameter A is not substantially smaller than the internal diameter B such that the cartridge 2 may be securely positioned on the peg 3 and the stopper 5 is being pushed at uniformly instead of at an edge.

The packaging 1 may comprise or consist of cardboard, plastics, sheet metal, wood or any other suitable material.

In alternative embodiments, the packaging 1 may have any other shape provided there is a suitable space for the peg 3, in particular such that the peg 3 may be positioned to point upwards in a use position, e.g. in or on a cover 1.2 or lid or on a bottom 1.1 of the packaging 1.

The arrangement may also be adapted to be used with cartridges having bodies with shapes other than circular cylindrical by respectively shaping the peg 3.

The term “drug” or “medicament”, as used herein, means a pharmaceutical formulation containing at least one pharmaceutically active compound,

wherein in one embodiment the pharmaceutically active compound has a molecular weight up to 1500 Da and/or is a peptide, a protein, a polysaccharide, a vaccine, a DNA, a RNA, an enzyme, an antibody or a fragment thereof, a hormone or an oligonucleotide, or a mixture of the above-mentioned pharmaceutically active compound,
wherein in a further embodiment the pharmaceutically active compound is useful for the treatment and/or prophylaxis of diabetes mellitus or complications associated with diabetes mellitus such as diabetic retinopathy, thromboembolism disorders such as deep vein or pulmonary thromboembolism, acute coronary syndrome (ACS), angina, myocardial infarction, cancer, macular degeneration, inflammation, hay fever, atherosclerosis and/or rheumatoid arthritis,
wherein in a further embodiment the pharmaceutically active compound comprises at least one peptide for the treatment and/or prophylaxis of diabetes mellitus or complications associated with diabetes mellitus such as diabetic retinopathy,
wherein in a further embodiment the pharmaceutically active compound comprises at least one human insulin or a human insulin analogue or derivative, glucagon-like peptide (GLP-1) or an analogue or derivative thereof, or exendin-3 or exendin-4 or an analogue or derivative of exendin-3 or exendin-4.

Insulin analogues are for example Gly(A21), Arg(B31), Arg(B32) human insulin; Lys(B3), Glu(B29) human insulin; Lys(B28), Pro(B29) human insulin; Asp(B28) human insulin; human insulin, wherein proline in position B28 is replaced by Asp, Lys, Leu, Val or Ala and wherein in position B29 Lys may be replaced by Pro; Ala(B26) human insulin; Des(B28-B30) human insulin; Des(B27) human insulin and Des(B30) human insulin.

Insulin derivates are for example B29-N-myristoyl-des(B30) human insulin; B29-N-palmitoyl-des(B30) human insulin; B29-N-myristoyl human insulin; B29-N-palmitoyl human insulin; B28-N-myristoyl LysB28ProB29 human insulin; B28-N-palmitoyl-LysB28ProB29 human insulin; B30-N-myristoyl-ThrB29LysB30 human insulin; B30-N-palmitoyl-ThrB29LysB30 human insulin; B29-N—(N-palmitoyl-Y-glutamyl)-des(B30) human insulin; B29-N—(N-lithocholyl-Y-glutamyl)-des(B30) human insulin; B29-N-(ω-carboxyheptadecanoyl)-des(B30) human insulin and B29-N-(ω-carboxyheptadecanoyl) human insulin.

Exendin-4 for example means Exendin-4(1-39), a peptide of the sequence H-His-Gly-Glu-Gly-Thr-Phe-Thr-Ser-Asp-Leu-Ser-Lys-Gln-Met-Glu-Glu-Glu-Ala-Val-Arg-Leu-Phe-Ile-Glu-Trp-Leu-Lys-Asn-Gly-Gly-Pro-Ser-Ser-Gly-Ala-Pro-Pro-Pro-Ser-NH2.

Exendin-4 derivatives are for example selected from the following list of compounds:

### H-(Lys)4-des Pro36, des Pro37 Exendin-4(1-39)-NH2,

### H-(Lys)5-des Pro36, des Pro37 Exendin-4(1-39)-NH2,

### des Pro36 Exendin-4(1-39),

### des Pro36 [Asp28] Exendin-4(1-39),

### des Pro36 [IsoAsp28] Exendin-4(1-39),

### des Pro36 [Met(O)14, Asp28] Exendin-4(1-39),

### des Pro36 [Met(O)14, IsoAsp28] Exendin-4(1-39),

### des Pro36 [Trp(O2)25, Asp28] Exendin-4(1-39),

### des Pro36 [Trp(O2)25, IsoAsp28] Exendin-4(1-39),

### des Pro36 [Met(O)14 Trp(O2)25, Asp28] Exendin-4(1-39),

### des Pro36 [Met(O)14 Trp(O2)25, IsoAsp28] Exendin-4(1-39); or

### des Pro36 [Asp28] Exendin-4(1-39),

### des Pro36 [IsoAsp28] Exendin-4(1-39),

### des Pro36 [Met(O)14, Asp28] Exendin-4(1-39),

### des Pro36 [Met(O)14, IsoAsp28] Exendin-4(1-39),

### des Pro36 [Trp(O2)25, Asp28] Exendin-4(1-39),

### des Pro36 [Trp(O2)25, IsoAsp28] Exendin-4(1-39),

### des Pro36 [Met(O)14 Trp(O2)25, Asp28] Exendin-4(1-39),

### des Pro36 [Met(O)14 Trp(O2)25, IsoAsp28] Exendin-4(1-39),

wherein the group -Lys6-NH2 may be bound to the C-terminus of the Exendin-4 derivative;
or an Exendin-4 derivative of the sequence

### des Pro36 Exendin-4(1-39)-Lys6-NH2 (AVE0010),

### H-(Lys)6-des Pro36 [Asp28] Exendin-4(1-39)-Lys6-NH2,

### des Asp28 Pro36, Pro37, Pro38Exendin-4(1-39)-NH2,

### H-(Lys)6-des Pro36, Pro38 [Asp28] Exendin-4(1-39)-NH2,

### H-Asn-(Glu)5des Pro36, Pro37, Pro38 [Asp28] Exendin-4(1-39)-NH2,

### des Pro36, Pro37, Pro38 [Asp28] Exendin-4(1-39)-(Lys)6-NH2,

### H-(Lys)6-des Pro36, Pro37, Pro38 [Asp28] Exendin-4(1-39)-(Lys)6-NH2,

### H-Asn-(Glu)5-des Pro36, Pro37, Pro38 [Asp28] Exendin-4(1-39)-(Lys)6-NH2,

### H-(Lys)6-des Pro36 [Trp(O2)25, Asp28] Exendin-4(1-39)-Lys6-NH2,

### H-des Asp28 Pro36, Pro37, Pro38 [Trp(O2)25] Exendin-4(1-39)-NH2,

### H-(Lys)6-des Pro36, Pro37, Pro38 [Trp(O2)25, Asp28] Exendin-4(1-39)-NH2,

### H-Asn-(Glu)5-des Pro36, Pro37, Pro38 [Trp(O2)25, Asp28] Exendin-4(1-39)-NH2,

### des Pro36, Pro37, Pro38 [Trp(O2)25, Asp28] Exendin-4(1-39)-(Lys)6-NH2,

### H-(Lys)6-des Pro36, Pro37, Pro38 [Trp(O2)25, Asp28] Exendin-4(1-39)-(Lys)6-NH2,

### H-Asn-(Glu)5-des Pro36, Pro37, Pro38 [Trp(O2)25, Asp28] Exendin-4(1-39)-(Lys)6-NH2,

### H-(Lys)6-des Pro36 [Met(O)14, Asp28] Exendin-4(1-39)-Lys6-NH2,

### des Met(O)14 Asp28 Pro36, Pro37, Pro38 Exendin-4(1-39)-NH2,

### H-(Lys)6-desPro36, Pro37, Pro38 [Met(O)14, Asp28] Exendin-4(1-39)-NH2,

### H-Asn-(Glu)5-des Pro36, Pro37, Pro38 [Met(O)14, Asp28] Exendin-4(1-39)-NH2,

### des Pro36, Pro37, Pro38 [Met(O)14, Asp28] Exendin-4(1-39)-(Lys)6-NH2,

### H-(Lys)6-des Pro36, Pro37, Pro38 [Met(O)14, Asp28] Exendin-4(1-39)-(Lys)6-NH2,

### H-Asn-(Glu)5 des Pro36, Pro37, Pro38 [Met(O)14, Asp28] Exendin-4(1-39)-(Lys)6-NH2,

### H-Lys6-des Pro36 [Met(O)14, Trp(O2)25, Asp28] Exendin-4(1-39)-Lys6-NH2,

### H-des Asp28 Pro36, Pro37, Pro38 [Met(O)14, Trp(O2)25] Exendin-4(1-39)-NH2,

### H-(Lys)6-des Pro36, Pro37, Pro38 [Met(O)14, Asp28] Exendin-4(1-39)-NH2,

### H-Asn-(Glu)5-des Pro36, Pro37, Pro38 [Met(O)14, Trp(O2)25, Asp28] Exendin-4(1-39)-NH2,

### des Pro36, Pro37, Pro38 [Met(O)14, Trp(O2)25, Asp28] Exendin-4(1-39)-(Lys)6-NH2,

### H-(Lys)6-des Pro36, Pro37, Pro38 [Met(O)14, Trp(O2)25, Asp28] Exendin-4(S1-39)-(Lys)6-NH2,

### H-Asn-(Glu)5-des Pro36, Pro37, Pro38 [Met(O)14, Trp(O2)25, Asp28] Exendin-4(1-39)-(Lys)6-NH2;

or a pharmaceutically acceptable salt or solvate of any one of the afore-mentioned Exendin-4 derivative.

Hormones are for example hypophysis hormones or hypothalamus hormones or regulatory active peptides and their antagonists as listed in Rote Liste, ed. 2008, Chapter 50, such as Gonadotropine (Follitropin, Lutropin, Choriongonadotropin, Menotropin), Somatropine (Somatropin), Desmopressin, Terlipressin, Gonadorelin, Triptorelin, Leuprorelin, Buserelin, Nafarelin, Goserelin.

A polysaccharide is for example a glucosaminoglycane, a hyaluronic acid, a heparin, a low molecular weight heparin or an ultra low molecular weight heparin or a derivative thereof, or a sulphated, e.g. a poly-sulphated form of the above-mentioned polysaccharides, and/or a pharmaceutically acceptable salt thereof. An example of a pharmaceutically acceptable salt of a poly-sulphated low molecular weight heparin is enoxaparin sodium.

Antibodies are globular plasma proteins (˜150 kDa) that are also known as immunoglobulins which share a basic structure. As they have sugar chains added to amino acid residues, they are glycoproteins. The basic functional unit of each antibody is an immunoglobulin (Ig) monomer (containing only one Ig unit); secreted antibodies can also be dimeric with two Ig units as with IgA, tetrameric with four Ig units like teleost fish IgM, or pentameric with five Ig units, like mammalian IgM.

The Ig monomer is a “Y”-shaped molecule that consists of four polypeptide chains; two identical heavy chains and two identical light chains connected by disulfide bonds between cysteine residues. Each heavy chain is about 440 amino acids long; each light chain is about 220 amino acids long. Heavy and light chains each contain intrachain disulfide bonds which stabilize their folding. Each chain is composed of structural domains called Ig domains. These domains contain about 70-110 amino acids and are classified into different categories (for example, variable or V, and constant or C) according to their size and function. They have a characteristic immunoglobulin fold in which two β sheets create a “sandwich” shape, held together by interactions between conserved cysteines and other charged amino acids.

There are five types of mammalian Ig heavy chain denoted by α, δ, ε, γ, and μ. The type of heavy chain present defines the isotype of antibody; these chains are found in IgA, IgD, IgE, IgG, and IgM antibodies, respectively.

Distinct heavy chains differ in size and composition; α and γ contain approximately 450 amino acids and δ approximately 500 amino acids, while μ and ε have approximately 550 amino acids. Each heavy chain has two regions, the constant region (CH) and the variable region (VH). In one species, the constant region is essentially identical in all antibodies of the same isotype, but differs in antibodies of different isotypes. Heavy chains γ, α and δ have a constant region composed of three tandem Ig domains, and a hinge region for added flexibility; heavy chains μ and ε have a constant region composed of four immunoglobulin domains. The variable region of the heavy chain differs in antibodies produced by different B cells, but is the same for all antibodies produced by a single B cell or B cell clone. The variable region of each heavy chain is approximately 110 amino acids long and is composed of a single Ig domain.

In mammals, there are two types of immunoglobulin light chain denoted by λ and κ. A light chain has two successive domains: one constant domain (CL) and one variable domain (VL). The approximate length of a light chain is 211 to 217 amino acids. Each antibody contains two light chains that are always identical; only one type of light chain, κ or λ, is present per antibody in mammals.

Although the general structure of all antibodies is very similar, the unique property of a given antibody is determined by the variable (V) regions, as detailed above. More specifically, variable loops, three on the light (VL) and three on the heavy (VH) chain, are responsible for binding to the antigen, i.e. for its antigen specificity. These loops are referred to as the Complementarity Determining Regions (CDRs). Because CDRs from both VH and VL domains contribute to the antigen-binding site, it is the combination of the heavy and the light chains, and not either alone, that determines the final antigen specificity.

An “antibody fragment” contains at least one antigen binding fragment as defined above, and exhibits essentially the same function and specificity as the complete antibody of which the fragment is derived from. Limited proteolytic digestion with papain cleaves the Ig prototype into three fragments. Two identical amino terminal fragments, each containing one entire L chain and about half an H chain, are the antigen binding fragments (Fab). The third fragment, similar in size but containing the carboxyl terminal half of both heavy chains with their interchain disulfide bond, is the crystalizable fragment (Fc). The Fc contains carbohydrates, complement-binding, and FcR-binding sites. Limited pepsin digestion yields a single F(ab′)2 fragment containing both Fab pieces and the hinge region, including the H—H interchain disulfide bond. F(ab′)2 is divalent for antigen binding. The disulfide bond of F(ab′)2 may be cleaved in order to obtain Fab′. Moreover, the variable regions of the heavy and light chains can be fused together to form a single chain variable fragment (scFv).

Pharmaceutically acceptable salts are for example acid addition salts and basic salts. Acid addition salts are e.g. HCl or HBr salts. Basic salts are e.g. salts having a cation selected from alkali or alkaline, e.g. Na+, or K+, or Ca2+, or an ammonium ion N+(R1)(R2)(R3)(R4), wherein R1 to R4 independently of each other mean: hydrogen, an optionally substituted C1-C6-alkyl group, an optionally substituted C2-C6-alkenyl group, an optionally substituted C6-C10-aryl group, or an optionally substituted C6-C10-heteroaryl group. Further examples of pharmaceutically acceptable salts are described in “Remington's Pharmaceutical Sciences” 17. ed. Alfonso R. Gennaro (Ed.), Mark Publishing Company, Easton, Pa., U.S.A., 1985 and in Encyclopedia of Pharmaceutical Technology.

Pharmaceutically acceptable solvates are for example hydrates.

Those of skill in the art will understand that modifications (additions and/or removals) of various components of the apparatuses, methods and/or systems and embodiments described herein may be made without departing from the full scope and spirit of the present invention, which encompass such modifications and any and all equivalents thereof.

### LIST OF REFERENCES

1 packaging
1.1 bottom
1.2 cover
1.3 side wall
1.4 side wall
1.5 side wall
1.6 side wall
1.7 side wall
2 drug cartridge
2.1 body
2.2 front wall
2.3 rear end
3 peg
4 tubing
5 stopper
6 cannula
A diameter
B internal diameter
D movement
E offset distance
H height
V volume

1-15. (canceled)

16. An arrangement for priming a drug cartridge comprising a body to which a tubing can be fitted, the body having an open rear end, wherein a stopper is slidably disposed within the body, the arrangement comprising a packaging for a number of drug cartridges, wherein a peg is arranged on the packaging, the peg having a height which is such that when a cartridge is being put on the peg with the rear end and pushed against it until the rear end abuts a surface of the packaging, the stopper is moved within the body by such a distance that a predetermined volume of a medicament is displaced from the cartridge.

17. The arrangement according to claim 16, wherein the peg has a diameter at most as large as an internal diameter of the body.

18. The arrangement according to claim 17, wherein the diameter of the peg is smaller than the internal diameter.

19. The arrangement according to claim 16, wherein the predetermined volume of medicament is at least as large as an internal volume of the tubing.

20. The arrangement according to claim 16, wherein a movement of the stopper required to displace the predetermined volume is given by the equation
$$D = \frac{4 V}{π {B}^{2}} ,$$
wherein the height of the peg is substantially equal to the movement.

21. The arrangement according to claim 16, wherein a movement of the stopper required to displace the predetermined volume is given by the equation
$$D = \frac{4 V}{π {B}^{2}} ,$$
wherein the height of the peg is slightly larger than the movement.

22. The arrangement according to claim 20, wherein an offset distance, by which the stopper is offset from the rear end of the body, combined with a movement distance of the stopper is equal to the height.

23. The arrangement according to claim 16, wherein the peg has a circular cylindrical shape.

24. The arrangement according to claim 16, wherein the peg has one or more vent openings or notches.

25. The arrangement according to claim 16, wherein the peg has a non-circular cylindrical or prismatic shape.

26. The arrangement according to claim 16, wherein the packaging comprises one or more of cardboard, plastics, sheet metal and wood.

27. The arrangement according to claim 16, wherein the peg is positioned on the packaging to point upwards in a use position.

28. The arrangement according to claim 16, wherein the packaging has the shape of a box with a bottom, a cover and side walls.

29. The arrangement according to claim 28, wherein the peg is arranged on the cover or on the bottom of the packaging.

30. The arrangement according to claim 29, wherein the peg is arranged on an inner surface of the cover of the packaging.

31. The arrangement of claim 16, wherein the drug cartridge contains medicament.

32. A method for priming a drug cartridge using an arrangement, wherein the drug cartridge comprises a body to which a tubing can be fitted, the body having an open rear end, wherein a stopper is slidably disposed within the body and the arrangement comprises a packaging for a number of drug cartridges, wherein a peg is arranged on the packaging, the peg having a height which is such that when a cartridge is being put on the peg with the rear end and pushed against it until the rear end abuts a surface of the packaging, the stopper is moved within the body by such a distance that a predetermined volume of a medicament is displaced from the cartridge, the method comprising:
orienting the drug cartridge such that the stopper is in contact with the peg on the packaging and;
applying a force on the drug cartridge towards the peg, such that the stopper is moved further into the body of the drug cartridge.

33. The method of claim 32, further comprising connecting a tube to the body of the drug cartridge prior to applying the force on the drug cartridge.
